(19) United States
(12) Patent Application Publication
(10) Pub. No.: US 2009/0324522 A1
(43) Pub. Date: Dec. 31, 2009

(54) SKIN PROTECTANT COMPOSITIONS

(75) Inventor: Nathalie Chevreau, Salt Lake City, UT (US)

Correspondence Address: TRASKBRITT, P.C., P.O. BOX 2550, SALT LAKE CITY, UT, 84110, US

(73) Assignee: WESTERN HOLDINGS, LLC, Carson City, NV (US)

(21) Appl. No.: 12/487,526

(22) Filed: Jun. 18, 2009

Related U.S. Application Data
(60) Provisional application No. 61/073,720, filed on Jun. 18, 2008.

(51) Int. Cl. A61K 8/97

(52) U.S. Cl. 424/59

(57) ABSTRACT

A topical composition for the protection of skin from damage caused by ultraviolet radiation. The composition includes a direct antioxidant and a phase 2 enzyme inducer (indirect antioxidant) in cosmetic and dermopharmaceutical compositions that protects and prevents skin damage and aging caused by UV radiation. In a particular embodiment, the composition includes a broccoli extract in combination with a composition including Hibiscus flower extract, Ferula Assa Foetida root extract, pear fruit extract, and green tea leaf extract, which exhibit a synergistic effect in protecting the skin from the adverse effects of ultraviolet radiation. In another embodiment, the composition further includes Vitamin C and Vitamin E. The antioxidant compositions can be incorporated into cosmetics, pharmaceutical compositions, sunscreen products, moisturizing lotions, skin toners, and other skin care products.

### CROSS-REFERENCE TO RELATED APPLICATION

This application claims the benefit under 35 U.S.C. § 119(e) of U.S. Provisional Application No. 61/073,720, filed Jun. 18, 2008, which is incorporated herein by reference.

### BACKGROUND OF THE INVENTION

1. Field of the Invention

This invention relates to topical antioxidant compositions for the protection and treatment of human skin, particularly skin exposed to harmful ultraviolet radiation.

2. State of the Art

The ultraviolet (UV) wavelengths of sunlight can cause sunburn (erythema) and blistering (edema). Exposure to ultraviolet light can also cause the skin to feel dry and taut in moderate doses, and to peel if exposed to higher doses. These acute or short term effects are readily perceptible. However, other acute effects, such as photo-immunosuppression, cross-linking of deoxyribonucleic acid (DNA), DNA breakage and mutations, formation of sunburn cells, and loss of Langerhans cells, are not as readily discernable. More serious long term effects include skin cancer and premature aging of the skin.

Sunscreen products are known to protect the skin from some of the harmful effects of ultraviolet light exposure. These products contain molecules that absorb the harmful wavelengths of ultraviolet light before they can reach or be absorbed in the skin. The absorbed light/energy reacts with skin components to be dissipated in the skin and environment, which allows these molecules to revert to a lower energy state, and subsequently absorb another photon of light. In this manner, sunscreen agents can absorb numerous photons of ultraviolet light in a relatively short period of time. By absorbing the harmful wavelengths of light, sunscreen products prevent many of the acute and chronic effects caused by ultraviolet light.

However, sunscreen products are not perfect in their mode of action. There is no single sunscreen agent that is capable of absorbing all of the harmful wavelengths striking the skin. Higher Sun Protection Factor (SPF) formulations address this problem by including a combination of sunscreen agents in the formulation. However, even when using a combination of sunscreen agents, these products do not provide complete protection, particularly from the longer ultraviolet wavelengths known as UVA radiation in the range of 315-400 nm, which makes up about 90% of the UV radiation from sun rays. These longer UVA wavelengths and UVB wavelengths create free radicals, reactive oxygen species (ROS) and electrophiles (electron acceptor molecules, such as free radical scavengers) by reacting with skin components. These free radicals, ROS and electrophiles are believed to be responsible for the premature aging of the skin commonly linked to ultraviolet light exposure, as well as causing sunburn keratinocytes, cell death, inflammation, immune suppression, protein (collagen, elastin) damage, DNA breakage and mutation, and cancer.

ROS include singlet oxygen, the superoxide radical, peroxyl radical, hydrogen peroxide, and the hydroxyl radical, as well as the reaction products produced by these free radicals. Due to their reactivity, ROS relatively indiscriminately react with other molecules, and generate a cascade of harmful free radical reactions in the skin.

The skin possesses defense mechanisms against the generation of ROS. These defenses include the presence of enzymes such as superoxide dismutase, catalase, glutathione transferase, glutathione peroxidase and glutathione reductase, as well as antioxidants such as tocopherols, ubiquinone, ubiquinol, ascorbic acid and dehydroascorbic acid. Unfortunately, ultraviolet light entering the skin can easily overwhelm these defense systems, such that the amount of superoxide dismutase and glutathione transferase in the skin declines significantly upon irradiation with solar simulated ultraviolet light. Simultaneous with the loss of these reducing enzymes, there is a dramatic increase in lipid peroxidation of the linoleates present in cell membranes. There is also an increase in thiobarbituric acid reactive substances present in the skin, which represent a collection of molecules that are formed from ROS.

Since sunscreens are unable to completely protect the skin against the adverse effects of ultraviolet radiation, alternative modes of protection have been proposed. Vitamins, such as Vitamin E acetate, have been shown to make the skin softer and smoother after topical application, which can offset some of the damaging effects of the sun. Vitamin A palmitate has been shown to create smoother skin and help enhance the process of cellular turnover. This enhancement rids the skin of the outermost dead layer of skin by bringing more youthful appearing skin cells to the surface. Other materials, such as hyaluronic acid and pyrrolidone carboxylic acid (PCA), have also been used for their ability to enhance the moisture binding capacity of the skin and therefore lead to smoother, softer skin.

In spite of advances in recent years in the protection of skin from harmful ultraviolet radiation, the epidemic of skin cancer and skin damage from the effects of this radiation has continued unabated. The loss of portions of the ozone layer from environmental pollution is believed to have contributed to an increase in ambient ultraviolet radiation that reaches exposed skin. Many skin protection preparations that could prevent sun damage have an unacceptable odor or texture that discourages their more frequent use, and many of the available skin protectants do not sufficiently protect the skin from these many mechanisms of injury. Hence there is a significant need for commercially acceptable or improved preparations that can be topically applied to human and animal skin to offset the harmful effects of ultraviolet radiation.

### BRIEF SUMMARY OF THE INVENTION

One embodiment of the present invention includes a composition and method for inhibiting skin damage induced by ultraviolet radiation, by applying topically to the skin a cytoprotective composition which includes broccoli sprout extract (“BSE”) in a sufficient amount to protect the skin from damaging effects of ultraviolet radiation. In particular embodiments, the composition can further include wasabi sprout extract (“WSE”). In other embodiments, the composition can further include Hibiscus flower extract, Ferula Assa Foetida root extract, pear fruit extract, and green tea leaf extract (collectively “HFPGE”), which exhibit a synergistic effect in protecting the skin from the adverse effects of ultraviolet radiation when used in combination with BSE. In another embodiment, the composition may further include Vitamin C and Vitamin E. In another embodiment, the composition may include CoQ10, vitamin A and its derivatives, and carotenoids

Methods of protecting the skin from UVB/UVA radiation damage include providing a composition comprising BSE and at least one other skin protectant or antioxidant, and applying the composition to the skin. The other skin protectant or antioxidant can include WSE, HFPGE, Vitamin C, Vitamin E, and/or other skin care actives.

The composition of the present invention may be provided in an aqueous or non-aqueous solution, suspension or an emulsion (water-in-oil or oil-in-water). The composition may be a skin toner composition, a moisturizing lotion, a sunscreen composition, a skin cleanser, or any other skin treatment composition. The composition may also be used in methods of protecting skin against the harmful effects of ultraviolet radiation, by applying topically to the skin an amount of the composition effective to reduce the UVB/UVA-induced damage in the skin. The composition may be applied before or after exposure to the sun, but is preferably applied prior to sun exposure, for example immediately before sun exposure.

### DETAILED DESCRIPTION OF THE INVENTION

Given the known effects of ultraviolet radiation on the skin and the inadequacy of many present skin protectants to interfere with the mechanisms of cellular damage, there is a need for effective alternative products that provide protection from the harmful effects of ultraviolet light. The present invention achieves these objectives by combining several antioxidants/electrophiles in a consumer acceptable form, which at the same time very effectively mitigates the damaging effects of sunlight on the skin. Additionally, the combination of antioxidants/electrophiles in the present composition provides unexpectedly superior protection against the damaging effects of ultraviolet light exposure to that provided by the individual antioxidants, as shown in the Examples below.

One embodiment of the present invention includes a composition and method for inhibiting skin damage induced by ultraviolet radiation, by applying topically to the skin an antioxidant/electrophiles composition which includes BSE in a sufficient amount to protect the skin from damaging effects of ultraviolet radiation, particularly UVA radiation. In particular embodiments, the composition includes BSE and WSE. In alternative embodiments, the composition includes BSE and HFPGE, which exhibit a synergistic effect in protecting the skin from the adverse effects of ultraviolet radiation when used in combination with BSE. Optionally, the BSE and HFPGE composition can include WSE. In another embodiment, the composition may further include Vitamin C and Vitamin E.

Methods of protecting the skin from UVB/UVA radiation damage include providing a composition comprising BSE and at least one other skin protectant or antioxidant, and applying the composition to the skin. The other skin protectant or antioxidant can include WSE, HFPGE, Vitamin C, Vitamin E, and/or other skin care actives.

In particular embodiments, the composition includes BSE in an amount from about 2 to about 3 weight percent (% w/w). In particular embodiments, the composition includes sulfurophanes (organic and synthetic) and derivatives thereof in an amount from about 10 nm or greater. The composition may further include WSE in an amount from about 0.1 to about 1 weight percent. Alternative compositions may further include HFPGE in an amount from about 2 to about 3 weight percent.

In a particular embodiment of the invention, the HFPGE composition includes Hibiscus sabdariffa Flower Extract in an amount from about 2% to about 4%, Ferula assa Foetida Root Extract in an amount from about 2 to about 4%, Pyrus communis (Pear) Fruit Extract in an amount from about 3 to about 5%, and Camellia sinensis (Green Tea) Leaf Extract in an amount from about 5 to about 7%.

### Additional Skin Care Actives

The compositions of the present invention contain at least one additional skin care active. The compositions of the present invention may contain additional skin care actives as well. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, functional isolates (peptides, botanical extracts, marine extracts, protein hydrolases, etc.) and vitamins and derivatives thereof.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

### Chelators

The compositions of the present invention may also contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage.

### Polyphenols

The compositions of the present invention may optionally contain a flavonoid compound. Flavonoids are broadly disclosed in U.S. Pat. Nos. 5,686,082 and 5,686,367, both of which are incorporated by reference herein. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof, flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof, chromones selected from unsubstituted chromones, mono-substituted chromones, di-substituted chromones, and mixtures thereof, one or more dicoumarols; one or more chromanones; one or more chromanols; isomers (e.g., cis/trans isomers) thereof, and mixtures thereof. By the term “substituted” as used herein means flavonoids wherein one or more hydrogen atom of the flavonoid has been independently replaced with hydroxyl, C₁-C₈ alkyl, C₁-C₄ alkoxyl, O-glycoside, and the like or a mixture of these substituents.

Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted trans-chalcone), mono-hydroxy chalcones (e.g., 2′-hydroxy chalcone, 4′-hydroxy chalcone, etc.), di-hydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

### Skin Soothing and Skin Healing Actives

The compositions of the present invention may comprise a skin soothing or skin healing active. Skin soothing or skin healing actives suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing active may be added to the present composition.

### Sunscreen Actives

The compositions of the subject invention may optionally contain a sunscreen active. As used herein, “sunscreen active” includes both sunscreen agents and physical sunblocks. Suitable sunscreen actives may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition.

A wide variety of conventional organic sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one), terephthalylidene dicamphor sulfonic acid and 4-isopropyl-di-benzoylmethane.

Also particularly useful in the compositions are sunscreen actives such as those disclosed in U.S. Pat. No. 4,937,370 issued to Sabatelli on Jun. 26, 1990, and U.S. Pat. No. 4,999,186 issued to Sabatelli & Spimak on Mar. 12, 1991. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. Representative members of this class of sunscreening agents are 4-N,N-(2-ethylhexyl)methyl-amino benzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof.

A safe and effective amount of the organic sunscreen active is used, with exact amounts varying depending upon the sunscreen or sunscreens chosen and the desired Sun Protection Factor (SPF).

### Conditioning Agents

The compositions of the present invention may contain a conditioning agent selected from humectants, moisturizers, or skin conditioners. A variety of these materials can be employed and each can be present at any suitable level. These agents include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, glucosamine); hyaluronic acid; lactamide monoethanolamine; acetamide monoethanolamine; panthenol; allantoin; and mixtures thereof. Also useful herein are the propoxylated glycerols described in U.S. Pat. No. 4,976,953, to Orr et al, issued Dec. 11, 1990.

Also useful are various C₁-C₃₀ monoesters and polyesters of sugars and related materials. These esters are derived from a sugar or polyol moiety and one or more carboxylic acid moieties. Such ester materials are further described in, U.S. Pat. No. 2,831,854, U.S. Pat. No. 4,005,196, to Jandacek, issued Jan. 25, 1977; U.S. Pat. No. 4,005,195, to Jandacek, issued Jan. 25, 1977, U.S. Pat. No. 5,306,516, to Letton et al, issued Apr. 26, 1994; U.S. Pat. No. 5,306,515, to Letton et al, issued Apr. 26, 1994; U.S. Pat. No. 5,305,514, to Letton et al, issued Apr. 26, 1994; U.S. Pat. No. 4,797,300, to Jandacek et al, issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985.

### Structuring Agents

The compositions hereof, and especially the emulsions hereof, may contain a structuring agent.

Representative structuring agents of the present invention can be selected from stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from stearic acid, palmitic acid, stearyl alcohol, cetyl alcohol, behenyl alcohol, steareth-2, and mixtures thereof.

### Thickening Agent (Including Thickeners and Gelling Agents)

The compositions of the present invention can contain one or more thickening agent. Nonlimiting classes of thickening agents include those selected from carboxylic acid polymers, crosslinked polyacrylate polymers, polyacrylamide polymers, polysaccharides, and gums.

### Dermatologically-Acceptable Carrier

The topical compositions of the present invention also contain a dermatologically acceptable carrier. The phrase “dermatologically-acceptable carrier,” as used herein, means that the carrier is suitable for topical application to the keratinous tissue, has good aesthetic properties, is compatible with the actives of the present invention and any other components, and will not cause any untoward safety or toxicity concerns. A safe and effective amount of carrier is from about 50% to about 99.99%, preferably from about 80% to about 99.9%, more preferably from about 90% to about 98%, and even more preferably from about 90% to about 95% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein.

The carriers may contain an emulsion such as oil-in-water emulsions, water-in-oil emulsions, and water-in-silicone emulsions. As will be understood by the skilled artisan, a given component will distribute primarily into either the water or oil/silicone phase, depending on the water solubility/dispersbility of the component in the composition. Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.01% to about 10%, more preferably from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986).

The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending on the desired product form. Exemplary low viscosity emulsions have a viscosity of from about 1 centipoise to about 1,000,000 centipoise.

### Composition Preparation

The compositions useful for the methods of the present invention are generally prepared by conventional methods such as are known in the art of making topical compositions. Such methods typically involve mixing of the ingredients in one or more steps to a relatively uniform state, with or without heating, cooling, application of vacuum, and the like.

### EXAMPLES

### Example 1

### Antioxidant Compositions

Antioxidant activity for the antioxidant compositions are evaluated in mammals and humans. In vivo effects are studied for three particular compositions:
Formula 1: 3% HFPGE+0.5% vitamin C+0.5% vitamin E in a cream base
Formula 2: WSE and BSE in the cream base. 2.5% BSE and WSE containing isothiocyanate sulforaphane (1-isothiocyanato-(4R)-(methylsulfinyl)butane) ranging from 2000-5000 ppm. Minimum 200 nanoMoles active ITCs by weight in the finished product.
Formula 3: 3% HFPGE+0.5% vitamin C+0.5% vitamin E+2.5% WSE/BSE in the cream base
Placebo: cream base only
Control: nothing applied

### Example 2

### Preparation and Standardization of Broccoli Sprout Extracts

Broccoli (Brassica oleracea italica, cv. DeCicco) sprout extracts are prepared and hydrolyzed with daikon sprout myrosinase. The final preparations are dissolved in 80% acetone:20% water (v/v) and their isothiocyanate concentration (of which 90% is sulforaphane) is determined by the cyclocondensation reaction.

### Example 3

### Animals

Female SKH 1 hairless mice are maintained in a 12 h light/12 h dark cycle at 35% humidity and given free access to water and pelleted AlN 76A diet without antioxidants. Two groups of 9 month old mice are treated topically with either a single dose or three repeated doses at 24 h intervals of (a) 50 μL of broccoli sprout extract (in 80% acetone:20% water by volume) containing 0.5 umol of sulforaphane, applied to the caudal area of the back, thus delivering 100 nmol sulforaphane/cm2; and (b) 50 μL of vehicle, applied to the rostral area of the back. The animals are euthanized 24 h after the last dose. Two identical rectangular segments (1.5×1 cm) of dorsal skin within each treated area are removed: one is frozen in liquid N2 for analysis of NQO1 enzyme activity and the other is submerged in optimal cutting temperature compound and then frozen at −80° for histologic cryosectioning.

### Example 4

### Human Subjects

Healthy human subjects are recruited by advertising, screened, and skin punch biopsies are obtained. For a safety study, a circle (1 cm in diameter) is drawn on the skin of the forearm and the extract is applied to the center of the circle by using a positive displacement pipette. Two broccoli sprout extracts prepared in 80% acetone:20% water (v/v) that differed 10 fold in isothiocyanate concentration (2.7 and 26.2 mmol/L) are used. The single volumes of extract applied should not exceed 3.25 μL. To maintain localization, multiple applications are made and each is allowed to dry before applying the next. A maximum volume of 26 μL is applied at a single site. Each subject receives a placebo treated “spot” with the equivalent volume of the vehicle. Subjects are instructed not to wash the treated areas for at least 8 h after application and to return on two consecutive days for visual inspection. An investigator who is unaware of the treatment groups inspects and photographs the sites of application of extract or placebo. The next higher dose is applied only if there was no evidence of reaction to the previous one.

For the efficacy studies, subjects are instructed to maintain a low vegetable diet for two weeks, to refrain from consuming cruciferous vegetables and condiments, and to keep a food diary two days before and throughout the study. Broccoli sprout extracts or vehicle are applied topically to the center of 1 cm diameter circles drawn on the posterior waist region of the back either as single doses or three repetitive doses at 24 h intervals. This anatomic site is chosen because normally it is not exposed and there is a small risk of scarring due to the biopsies. Full thickness skin punch biopsies (3 mm diameter, maximum number of six per volunteer) are taken from treated and control sites 24 h after the last treatment, after s.c. application of lidocaine. The area of the biopsy is sutured and dressed. Specimens are immediately frozen in liquid N2 and stored at −80° C. until analyzed for phase 2 enzyme activity and anti-oxidant activity.

### Example 5

### NQO1 Enzyme Activity

Frozen skin tissue is pulverized in liquid N2 and the resulting powder is homogenized in 0.25 mol/L sucrose 10 mmol/L Tris HCl (pH 7.4). The clear supernatant fractions obtained after centrifugation at 14,000×g for 30 min at 4° C. are analyzed for protein concentration and enzyme activity levels.

### Example 5

### Immunohistochemistry

Frozen tissue blocks are sectioned by a microtome cryostat and the resulting cryosections (10 μm thickness) are mounted on microscope glass slides. Sections are immunostained using highly specific primary antibodies against NQO1 (1:200 dilution), GSTA1 (1:200 dilution), and HO-1 (1:1,000 dilution), followed by FITC conjugated secondary antibody. A microscope equipped with an excitation filter and a barrier filter is used to view the FITC fluorescence.

### Example 6

### Statistical Analysis

Comparisons of NQO1 activity (expressed as treated over control ratio) are evaluated on all doses, by subject, and across multiple subjects.

### Example 7

Measurement of UV Erythema

To evaluate UV-mediated damage of human skin, erythema measurement is performed as a noninvasive biomarker according to the protocol detailed in Talalay et al., Sulforaphane mobilizes cellular defenses that protect skin against damage by UV radiation, PNAS, Vol. 104 (No. 44), Oct. 30, 2007, the contents of which are hereby incorporated by reference in its entirety.

The present invention takes advantage of the surprising superiority found when combining two skin agents that protect the skin from ultraviolet radiation and, in particular UVA radiation, one agent from a class of direct antioxidants and the other from a class of indirect antioxidants (phase 2 enzyme inducers). The compositions of the invention can be applied to skin both before or after exposure to ultraviolet radiation, to provide the protective effect, however application before exposure to the sun is preferred. Daily applications of the skin protectant may be used, even if exposure to the sun is not anticipated, to diminish the aging effects of ROS in the skin.

As used in this specification, reducing damage caused by exposure to ultraviolet radiation means reducing damage as measured by the assays shown in the above Examples. Ultraviolet radiation refers to electromagnetic radiation having a wavelength shorter than the wavelengths of visible light and longer than those of x-rays. UVA radiation refers to radiation in the range of 315-400 nm and UVB radiation refers to radiation in the range of 280-315 nm. An antioxidant is a substance that opposes the effects of ROS, either by scavenging or reducing ROS or interfering with the production of ROS.

In view of the many possible embodiments to which the principles of my invention may be applied, it should be recognized that the illustrated embodiments are only specific examples of the invention and should not be taken as a limitation on the scope of the invention.

What is claimed is:

1. A topical composition for reducing skin damage induced by ultraviolet radiation, the composition comprising:
broccoli sprout extract in a sufficient amount to reduce the skin damage when the broccoli sprout extract is applied topically; and
at least one other skin protectant that reduces the skin damage caused by ultraviolet light.

2. The topical composition of claim 1, wherein the at least one other skin protectant is selected from the group consisting of wasabi sprout extract, green tea leaf extract, Vitamin C, Vitamin E, CoQ10, caratenoids, and other skin care actives.

3. The topical composition of claim 1, wherein the composition further comprises wasabi sprout extract.

4. The topical composition of claim 1, wherein the composition further comprises green tea leaf extract.

5. The topical composition of claim 1, wherein the composition comprises a suspension.

6. The topical composition of claim 1, wherein the composition comprises an emulsion.

7. The topical composition of claim 1, wherein the composition comprises a solution.

8. The topical composition of claim 1, wherein the composition comprises from about 2 to about 3 weight percent (% w/w) of broccoli sprout extract.

9. The topical composition of claim 3, wherein the composition comprises from about 0.1 to about 1 weight percent (% w/w) of wasabi sprout extract.

10. The topical composition of claim 4, wherein the composition comprises from about 2 to about 4 weight percent (% w/w) of green tea leaf extract.

11. The topical composition of claim 1, further comprising sulfurophanes.

12. The topical composition of claim 1, further comprising a sunscreen active.

13. A method of protecting the skin from UVB/UVA radiation damage comprising:
providing a composition comprising broccoli sprout extract and at least one other skin protectant or antioxidant; and
applying the composition to the skin.

14. The method of claim 13, wherein the at least one other skin protectant or antioxidant is selected from the group consisting of wasabi sprout extract, green tea leaf extract, Vitamin C, Vitamin E, and/or other skin care actives.

15. The method of claim 13, wherein the at least one other skin protectant is selected from the group consisting of wasabi sprout extract, green tea leaf extract, Vitamin C, Vitamin E, CoQ10, caratenoids, and other skin care actives.

16. The method of claim 13, wherein the composition further comprises wasabi sprout extract.

17. The method of claim 13, wherein the composition further comprises green tea leaf extract.

18. A topical composition for reducing skin damage induced by ultraviolet radiation, the composition comprising:
green tea sprout extract in a sufficient amount to reduce the skin damage when the broccoli sprout extract is applied topically; and
at least one other skin protectant that reduces the skin damage caused by ultraviolet light.

19. The topical composition of claim 18, further comprising broccoli sprout extract and wasabi sprout extract.

20. The topical composition of claim 18, further comprising vitamin C and vitamin E.
